(19) United States
(12) Patent Application Publication
(10) Pub. No.: US 2014/0378914 A1
(43) Pub. Date: Dec. 25, 2014

(54) Cannula Apparatus

(71) Applicants: Ziad Hassan Abu Ayyash, Amman (JO); Lourance Abdel-Razzaq Enad Alzyarah Al Hadid, Amman (JO)

(72) Inventors: Ziad Hassan Abu Ayyash, Amman (JO); Lourance Abdel-Razzaq Enad Alzyarah Al Hadid, Amman (JO)

(21) Appl. No.: 13/925,286

(22) Filed: Jun. 24, 2013

(51) Int. Cl. A61M 5/158

(52) U.S. Cl. CPC A61M 5/158; A61M 2005/1585; A61M 2005/1587
USPC 604/256

(57) ABSTRACT

The present invention relates to safe medical apparatuses, especially to cannulae that have safety means, and thus help health workers to provide care of patients in a safe manner by protecting such health workers and nurses from infections caused by contaminated blood of a patient. A cannula of the present invention reduces or eliminates the probability of any blood leaks, the probability that a needle body touches the nurse's skin, and the need to use a needle for administering medications. The use of the cannula of the present invention allows for better control over the needle. The cannula of the present invention is characterized by having a trunk with a rubber, a funnel, a valve with a rubber, and two plastic extensions extruded laterally, wherein each of said plastic extensions has a two-face sticker that allows the fixation of the cannula of the present invention on the patient's skin.

### FIELD OF THE INVENTION

The present invention relates to medical apparatus, more particularly to cannula apparatus that have safety features for avoiding the infection of nurses by a patient's blood.

### BACKGROUND OF THE INVENTION

The susceptibility of nurses and health workers to infection by patient's blood is a problem facing those health workers and nurses, especially when such infections result from accidental sticks by contaminated cannulae to such health workers and nurses. For this reason, numerous conventional solutions to this problem are found in the prior art.

Among these conventional solutions, there is provided an apparatus comprising multi-position stop-cock valves for use in the intravenous administration of different medications wherein the said apparatus is provided with integral color codes on the valve body and the valve handles whereby the valve position and the medication that flows through a particular valve will be immediately apparent to the attendant.

Another conventional solution discloses a guard for the tip of an intravenous needle which operates automatically when the needle is withdrawn from an intravenous catheter.

In the traditional and the prior art cannulae, the probability of blood spray while injecting the needle inside the vein, or while withdrawing such needle from a vein still exists. Thus, a safe cannula must be put in place such that it protects such health workers from being infected by contaminated blood.

### SUMMARY OF THE INVENTION

Therefore, it is an object of the present invention to have a safe medical apparatus which prevents health workers from being infected by patient's contaminated blood that, thereby, provides a comfortable way of taking care of patients.

It is an aspect of the present invention to have a novel safe cannula that eliminates the susceptibility of health workers and nurses to infections caused by contact with contaminated blood of a patient.

It is another aspect of the present invention to have a safe cannula that reduces or eliminates the probability of any blood leaks, the probability that the needle body touches the nurse's skin, and the need to use a needle for administering medications.

It is another aspect of the present invention to have a safe cannula that allows for better control over the needle.

It is another aspect of the present invention to have a safe cannula that allows for improved administration of intravenous injections to a patient.

It is another aspect of the present invention to have a safe cannula that allows for improved taking of blood samples from a patient.

The cannula of the present invention is characterized by having a trunk with a rubber, a funnel, a four-way valve with a rubber, and two plastic extensions extruded laterally, wherein each of said extensions has a two-face sticker that allows the fixation of the cannula of the present invention on the patient's skin.

In the cannula of the present invention, said trunk comprises a detachable sealed cylindrical longitudinal track in which the needle resides after the injection process is finished, wherein such trunk has a tiny protrusion at its rear end that narrows such track such that when the injection process is finished, the nurse or the health worker is able to force a plastic rear end of the needle into the narrowed area to prevent the return of the needle's sharp head to the outside of such trunk. When the needle is forced to reside in the narrowed area, the nurse detaches said trunk from the cannula body.

Said trunk rubber comprises a sealed rubber that prevents the undesirable back flow of the blood while said valve is open, wherein said sealed rubber secures the needle sharp head while the plastic end of the needle is held at a rear end of said trunk.

Accordingly, as a first aspect of the invention, there is provided a cannula apparatus comprising:
a first portion comprising a hollow body extending along a longitudinal axis adapted to receive a fluid tube and a needle for injection inside a mammalian body;
a second portion comprising a trunk extending along the longitudinal axis adapted to receive and lodge the needle;
a valve intermediate the first and second portions adapted to allow movement of the needle between the first and second portions and to block communication between the first and second portions when the needle is lodged inside the second portion for preventing penetration of fluid from the first portion inside the second portion.

The valve is preferably further adapted to enable an end-user of injecting a medical fluid inside the first portion for administration inside the mammalian body.

The valve is preferably further adapted to block communication between the first and second portions when the valve is used to receive the medical fluid for administration to the user.

The valve is preferably a four-way valve comprising a first and second groups of openings respectively for a first operation mode and a second operation mode, the first group of openings comprising a first opening and a second opening, and the second group of openings comprising a third opening and a fourth opening, wherein:
the first and second openings being in fluid communication with the end-user and fluid tube respectively for enabling the first operation mode comprising receiving the medical fluid from the end-user and enabling flow of the medical fluid inside the fluid tube for administration inside the mammalian body;
the third and fourth openings being adapted to be aligned along the longitudinal axis for enabling the second operation mode comprising enabling movement of the needle between the first and second portions during operation and retraction of the needle;
wherein the valve is adapted to prevent simultaneous use of the first and second operation modes.

Preferably, the valve is substantially cylindrical in shape and circular in cross-section for enabling an end-user of applying a circular motion on the valve for selecting between the first and second operation modes.

Preferably, the cylindrical valve has an upper edge, a lower edge and a peripheral wall surrounding the upper and lower edges, wherein the first opening is defined by the upper edge of the valve and the second, third and fourth openings are located at the peripheral wall of the valve.

Preferably, the third and fourth openings are collinear and adapted to be aligned along the longitudinal axis during the second operation mode and to be aligned transversally to the longitudinal axis during the first operation mode.

Preferably, the second portion further comprises a fluid insulator adapted to prevent undesired fluid from penetrating the second portion when the needle moves from the first portion inside the second portion.

Preferably, the fluid insulator is made of a suitable material for enabling the needle to pass through all in retaining the undesired fluid. The suitable material can be rubber.

Preferably, the second portion further comprises a funnel enclosing the fluid insulator and adapted to be connected to the valve.

Preferably, the funnel is adapted to securely enclose the head of the needle when lodged inside the second portion.

Preferably, the apparatus further comprises a needle handle adapted to manipulate the needle during operation.

Preferably, the second portion further comprises a stopper adapted to engage and retain the needle handle for preventing movement of the needle once lodged inside the second portion.

Preferably, the stopper comprises a narrow opening defined by the trunk within an end portion of the trunk.

Preferably, the apparatus further comprises support members adapted to support the apparatus on the mammalian body.

Preferably, the support members extend laterally from the apparatus and made of a resilient plastic material.

Preferably, the apparatus further comprises operation mode indicators located at the valve for enabling visualization and selection of the operation modes of the valve.

Preferably, the mammalian is a human.

The undesired fluid can be blood and the medical fluid can be IV fluid.

### BRIEF DESCRIPTION OF THE DRAWINGS

The invention will now be described with reference to the accompanying drawings, which illustrate a preferred embodiment of the present invention without restricting the scope of the invention's concept, and in which:

FIG. 1 illustrates a perspective view of a safe cannula configured according to a preferred embodiment of the present invention.

FIG. 2 illustrates a perspective view of a safe cannula's valve configured according to a preferred embodiment of the present invention.

FIG. 3 illustrates a front view of a safe cannula's valve configured according to a preferred embodiment of the present invention.

FIG. 4 illustrates a front view of a safe cannula's valve showing its internal configuration configured according to a preferred embodiment of the present invention.

### DETAILED DESCRIPTION OF THE PREFERRED EMBODIMENT

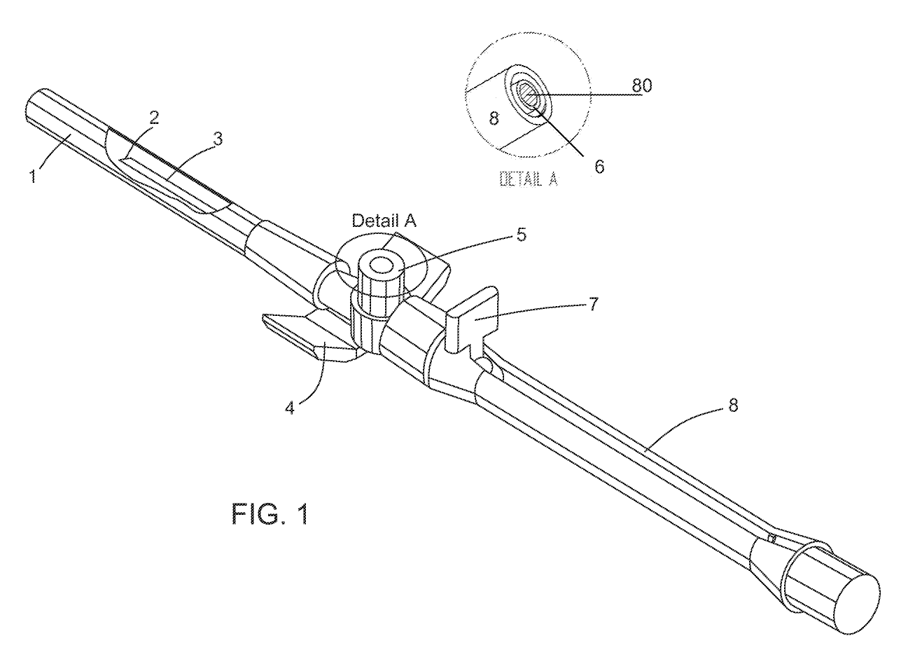

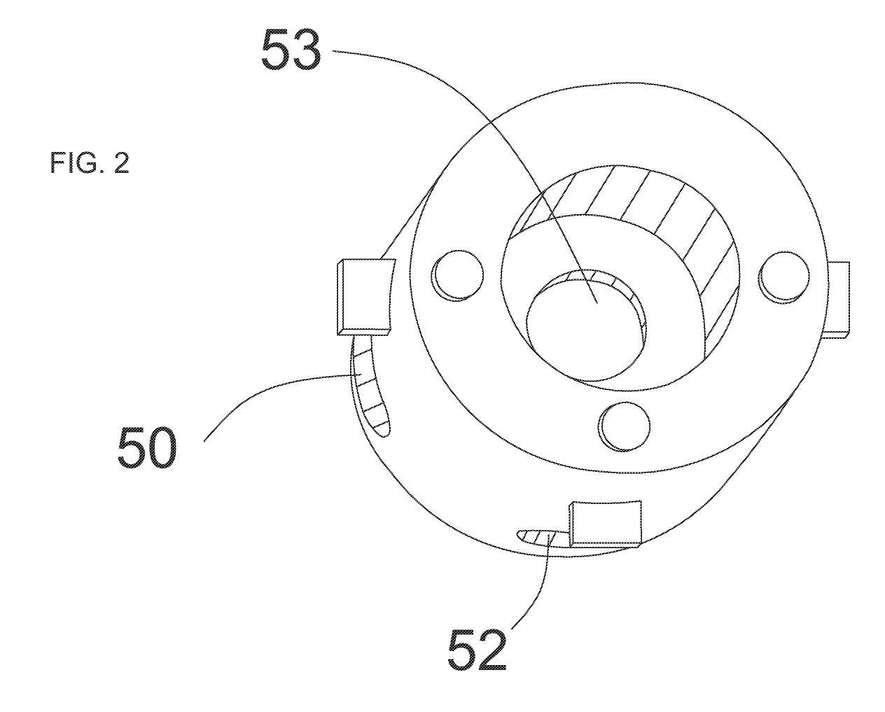

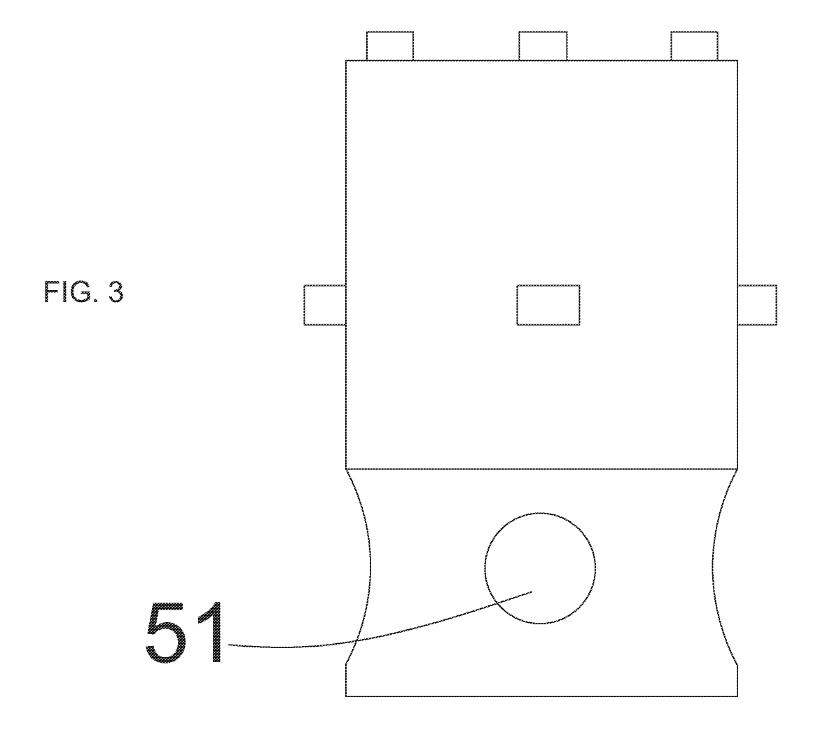

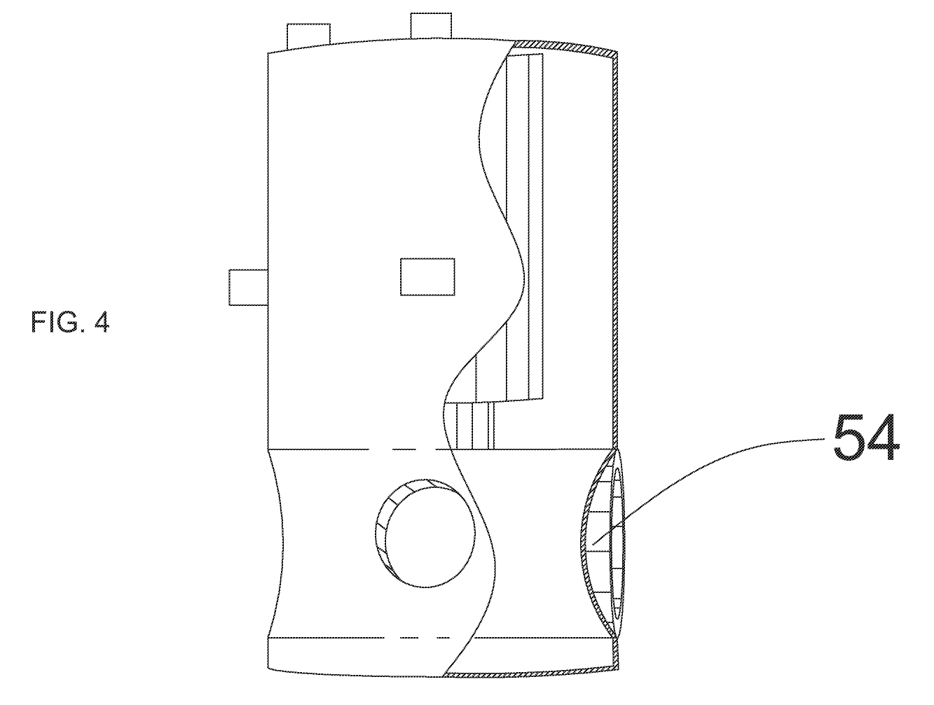

FIGS. 1, 2, 3, and 4 illustrate a safe cannula apparatus configured according to a preferred embodiment of the present invention. Referring to these figures the apparatus comprises a first portion and a second portion and a valve intermediate and in communication with the first and second portions.

The first portion of the apparatus comprises a hollow body, essentially comprising a needle cover 1 adapted to accommodate a fluid tube 3. The fluid tube 3 is preferably a flexible tube. The needle cover 1 extends longitudinally along a longitudinal axis along the first portion of the apparatus from a first end to a second end. The needle cover comprises an opening throughout the longitudinal axis between the first and second ends adapted to accommodate and lodge the fluid tube 3 between the two ends. The fluid tube is adapted to extend longitudinally along the longitudinal axis within the opening and to accommodate and lodge a needle between the first and second ends. The needle cover 1 is preferably made of plastic and is used to provide a cover to the needle; During operation, the needle 2 is injected into the patient's vein along with the accompanying fluid tube 3 such that the needle 2 can be pulled out of the patient's vein after the injection process, while the fluid tube 3 remains in the vein. The first end of the needle cover has an opening for allowing injection of the needle into the patient's body. The opening of the needle cover is preferably in the form of a channel. The fluid tube 3 is used to inject medical fluid inside the patient's body when the need is retracted.

The cannula apparatus preferably comprises two supporting members 4, made of a flexible material such as plastic or resilient paper, adapted to support the cannula apparatus on the patient's body for preventing movement of the apparatus during operation. For example, the supporting members can comprise two members having a two-face sticker that enables the fixation of such cannula apparatus on the patient's hand or arm, and thus preventing such cannula from moving which can be painful to said patient during operation;

The valve 5 of the present invention is preferably a four-way valve 5; wherein the valve is adapted to control the blood flow, stop the blood drain, control the medical fluid (e.g. IV fluid) supply during administration, maintain safety and control, and to allow the nurse to take blood samples in an easier and safer way than without use of said cannula as explained more extensively hereinafter.

The second portion of the apparatus comprises a trunk 8 extending longitudinally from a first end to a second end along the longitudinal axis, wherein the trunk is detachable from the cannula apparatus. The trunk 8 has a hollow body and is adapted to receive a funnel having a first funnel end and a second funnel end. The trunk 8 is adapted to receive the funnel 6 throughout a part of its length starting from the first end of the trunk such that the first end of the trunk corresponds substantially with the first end of the funnel. The trunk 6 further comprises an opening 82 extending along the longitudinal axis between the second funnel end and the second trunk end, the opening 82 being adapted to accommodate and lodge the needle for providing a safe housing for the needle when is being pulled out from the fluid tube 3 and first portion of the apparatus.

The needle 2 adapted to work with the present apparatus has a first end and second end extending along the longitudinal axis. The needle has a handle 7 at the second end for enabling the user to manipulate the needle operation. The handle 7 remains at all times within the second portion of the apparatus The trunk 8 has stopper 81 (in proximity of the second trunk end for stopping and retaining the needle handle for preventing the needle from sliding back or forth. The stopper can be in the form of a protrusion 81 adapted to engage with and retain the needle handle 7. The longitudinal opening 82 can also have a narrowed portion towards the second trunk end adapted such that when the injection process is finished, the nurse or the health worker forces the needle handle 7 into the narrowed portion to prevent the return of the needle's sharp head to the outside of such trunk that could occur if the needle handle 7 slides throughout the longitudinal axis along said opening 82 in the direction of the first trunk end. When the needle handle 7 engages with and is retained by in the narrowed area, the second portion of the apparatus and thus the trunk 8 can be detached from the cannula apparatus. The needle handle 7 is preferably made of plastic.

According to the present invention, the funnel 6 comprises fixing features for fixing the trunk 8 to the second portion of the apparatus, wherein such fixing features can comprise threads for example or any other suitable attachment means. The diameter of said funnel 6 is preferably smaller than the diameter of the trunk 8 and the space formed between the funnel 6 and the trunk 8 contains the fixing features (or attachment means). The second portion also comprises a fluid insulator 80 adapted to be located in said funnel 6, for preventing the blood and any other fluid from entering inside the second portion while allowing the need to slide through back/forth when in use and/or retraction mode. The fluid insulator 80 can be made of any suitable material adapted to enable passage of the needle all in retaining any fluid from passing through in both directions, such as rubber. The fluid insulator 80 prevents the undesirable back flow of the blood and any other fluid when the valve 5 is open and serves as a stopper to the needle sharp head when the y handle of the needle 7 is held in the narrowed portion of the trunk 8. The needle 2 is adapted to pass through the fluid insulator 80 in both directions for entering to the first portion of the apparatus through the valve 5 and backward inside the second portion of the apparatus while in retraction mode.

The apparatus comprises a valve 5, preferably a 4-way valve. The valve is located intermediate the first and second portions of the apparatus. The four-way valve 5 is preferably substantially cylindrical in shape having a valve axis corresponding to the cylinder axis. The valve 5 is thus substantially circular in cross-section. The valve 5 comprises a cylindrical peripheral wall 502 surrounding the upper and lower edges of the valve 5. The valve 5 defines a vertical valve opening 53 extending from the upper valve edge along the valve axis downwardly. The peripheral wall of the valve defines three lateral valve openings, a first lateral opening 50, a second lateral opening 51 and a third lateral opening 52. The lateral valve openings 50, 51, and 52 are adapted to control the blood and/or the medical fluid (e.g. IV fluid) flow to and from the patient's vein by circular twisting the valve, wherein two of these openings, the second and third openings 51 and 52, are collinear and are adapted to align along the longitudinal axis of the apparatus. The vertical opening 53 is used as an additional inlet for injecting a medical fluid into the patient's venous. The valve 5 is adapted to block fluid communication between the lateral openings 50, 51 & 52 and the vertical opening 53 in order to prevent backflow of the medical fluid and/or body fluid from the first portion of the apparatus to the end user through the vertical opening 53. Accordingly, the valve 5 also comprises a valve barrier 54, wherein such valve barrier 54 is preferably a T-shaped body made of rubber intersecting cylindrical-shaped tubes, wherein such valve barrier 54 connects between said collinear holes 51 and 52 and also creates a barrier between the vertical opening 53 and the other lateral openings. Said barrier 54 enables the nurse or the health worker to push any intravenous medication via vertical opening 53 into the patient's vein.

In fact, during the needle injection mode, the collinear lateral openings 51 and 52 must be aligned in order to enable the needle to extend through these lateral openings 51 and 52 along the longitudinal axis between the first and second portions of the apparatus. When the injection process of the needle is over, the injection is retracted and lodged inside the second portion of the apparatus as explained above. In this case, the valve 5 can be adjusted to close the communication between the first and second portions by a circular twisting motion of the valve 5 to have the openings 51 and 52 aligned transversally to the longitudinal axis of the apparatus. If a medical fluid is to be injected inside the vein through the fluid tube 3, the valve 5 is adjusted to have the first lateral opening 50 in fluid communication with the first portion of the apparatus. The medical fluid is therefore injected through the vertical valve opening 53 which is communicated to the flexible needle tube 3 through the first lateral valve opening 50. Since the second/third lateral valve openings 51 and 52 are in close position, the medical fluid and any other fluid inside the first portion of the apparatus (e.g. body) is prevented from entering into the second portion of the apparatus.

In the cannula of the present invention, said valve 5 also comprises a plurality of indicating members 55a, 55b, and 55c, wherein each of such members are positioned vertically above each of said lateral holes 50, 51, and 52 respectively. Said indicating members extend radially outwardly from the body of said valve 5, and gives indication about the status of the valve if being opened or closed. The default state of the valve is being opened with said collinear openings 51 and 52 are aligned with the longitudinal direction of the cannula of the present invention; wherein such alignment provides a path for the needle to leave the trunk 8 and pass through the cannula body in order to be injected into the patient's skin, and to leave the patient's skin after the injection process is finished in order to pass through the cannula body before reaching said trunk 8.

In the preferred embodiment of the present invention, the nurse should close said valve 5 by twisting such valve until said indicating members 55a and 55c are pointing in the perpendicular direction to the longitudinal direction of the cannula body (i.e. longitudinal axis of the apparatus) before detaching said trunk 8 in order to prevent any blood backflow outside the cannula body.

In the preferred embodiment of the present invention, the cannula body, the cover 1, the flexible tube 3, the valve 5, the funnel 6, the plastic end of the needle 7, and the trunk are made of plastic. Said trunk and valve rubbers are made of rubber.

While the invention has been described in detail and with reference to specific embodiments thereof, it will be apparent to one skilled in the art that various additions, omissions, and modifications can be made without departing from the spirit and scope thereof.

Although the above description contains many specificities, these should not be construed as limitations on the scope of the invention but is merely representative of the presently preferred embodiment of this invention. The embodiment of the invention described above is intended to be exemplary only. The scope of the invention is therefore intended to be limited solely by the scope of the appended claims.

1. A cannula apparatus comprising:
a first portion comprising a hollow body extending along a longitudinal axis adapted to receive a fluid tube and a needle for injection inside a mammalian body;
a second portion comprising a trunk extending along the longitudinal axis adapted to receive and lodge the needle;
a valve intermediate the first and second portions adapted to allow movement of the needle between the first and second portions and to block communication between the first and second portions when the needle is lodged inside the second portion for preventing penetration of fluid from the first portion inside the second portion.

2. The cannula apparatus as claimed in claim 1, wherein the valve is further adapted to enable an end-user of injecting a medical fluid inside the first portion for administration inside the mammalian body.

3. The cannula apparatus as claimed in claim 2, wherein the valve is adapted to block communication between the first and second portions when the valve is used to receive the medical fluid for administration to the user.

4. The cannula as claimed in claim 3, wherein the valve is a four-way valve comprising a first and second groups of openings respectively for a first operation mode and a second operation mode, the first group of openings comprising a first opening and a second opening, and the second group of openings comprising a third opening and a fourth opening, wherein:
the first and second openings being in fluid communication with the end-user and fluid tube respectively for enabling the first operation mode comprising receiving the medical fluid from the end-user and enabling flow of the medical fluid inside the fluid tube for administration inside the mammalian body;
the third and fourth openings being adapted to be aligned along the longitudinal axis for enabling the second operation mode comprising enabling movement of the needle between the first and second portions during operation and retraction of the needle;
wherein the valve is adapted to prevent simultaneous use of the first and second operation modes.

5. The cannula apparatus as claimed in claim 4, wherein the valve is substantially cylindrical in shape and circular in cross-section for enabling an end-user of applying a circular motion on the valve for selecting between the first and second operation modes.

6. The cannula apparatus as claimed in claim 5, wherein the cylindrical valve has an upper edge, a lower edge and a peripheral wall surrounding the upper and lower edges, wherein the first opening is defined by the upper edge of the valve and the second, third and fourth openings are located at the peripheral wall of the valve.

7. The cannula apparatus as claimed in claim 6, wherein the third and fourth openings are collinear and adapted to be aligned along the longitudinal axis during the second operation mode and to be aligned transversally to the longitudinal axis during the first operation mode.

8. The cannula apparatus as claimed in claim 7, wherein the second portion further comprises a fluid insulator adapted to prevent undesired fluid from penetrating the second portion when the needle moves from the first portion inside the second portion.

9. The cannula apparatus as claimed in claim 8, wherein the fluid insulator is made of a suitable material for enabling the needle to pass through all in retaining the undesired fluid.

10. The cannula apparatus as claimed in claim 9, wherein the suitable material is rubber.

11. The cannula apparatus as claimed in claim 10, wherein the second portion further comprises a funnel enclosing the fluid insulator and adapted to be connected to the valve.

12. The cannula apparatus as claimed in claim 11, wherein the funnel is adapted to securely enclose the head of the needle when lodged inside the second portion.

13. The cannula apparatus as claim in claim 12, further comprising a needle handle adapted to manipulate the needle during operation.

14. The cannula apparatus as claimed in claim 13, wherein the second portion further comprises a stopper adapted to engage and retain the needle handle for preventing movement of the needle once lodged inside the second portion.

15. The cannula apparatus as claimed in claim 14, wherein the stopper comprises a narrow opening defined by the trunk within an end portion of the trunk.

16. The cannula apparatus as claimed in claim 15, further comprising support members adapted to support the apparatus on the mammalian body.

17. The cannula apparatus as claimed in claim 16, wherein the support members extend laterally from the apparatus and made of a resilient plastic material.

18. The cannula apparatus as claimed in claim 17, further comprising operation mode indicators located at the valve for enabling visualization and selection of the operation modes of the valve.

19. The cannula apparatus as claimed in claim 1, wherein the mammalian is a human.

20. The cannula apparatus as claimed in claim 19, wherein the undesired fluid is blood and wherein the medical fluid is IV fluid.
